(19) United States
(12) Patent Application Publication
(10) Pub. No.: US 2010/0063464 A1
(43) Pub. Date: Mar. 11, 2010

(54) Tube Connector for a Vacuum Therapy Device

(76) Inventors: Johannes Meyer, Hamburg (DE); Dirk Baumann, Hamburg (DE); Janine Rohrs, Hamburg (DE)

Correspondence Address: NORTH WEBER & BAUGH LLP, 2479 E. BAYSHORE ROAD, SUITE 707, PALO ALTO, CA, 94303, US

(21) Appl. No.: 11/910,906

(22) PCT Filed: Mar. 28, 2006
(86) PCT No.: PCT/EP2006/002794

§ 371 Date: Nov. 16, 2009

(30) Foreign Application Priority Data
Apr. 6, 2005 (DE) 10 2005 015 878.1

(51) Int. Cl. A61M 1/00

(52) U.S. Cl. 604/319

(57) ABSTRACT

The invention relates to a therapeutic device to promote the healing of a wound, comprising a cover around the wound to form an essentially airtight cover over the wound, whereby a liquid-permeable pad is preferably provided between the wound and cover, a drainage line connecting the cover with a suction pump so that the wound can be suctioned to draw the fluids from it, and a connector to connect the cover with the drainage line, whereby the connector is provided with an opening to connect the proximal end of the drainage line to the wound, with which a more solid connection is guaranteed between the cover and the drainage line, which however at the same time has sufficient elasticity to conform to the respective body form and/or shape of the wound. It is also proposed that the connection contain fastening sections which extend from an area radiating from the opening to the area near the outer edge of the cover and which are separated from each other by notches.

### CROSS REFERENCE TO RELATED PATENT APPLICATIONS

This application is the National Phase Application under 35 USC §371 of International Application No. PCT/EP2006/002794, filed Mar. 28, 2006, which claims priority to German Patent Application 10 2005 015 878.1, filed Apr. 6, 2005, which is incorporated by reference in its entirety. This application is related to U.S. patent application Ser. No. 11/909,596, entitled “Vacuum Therapy Device”, filed Sep. 24, 2007.

### BACKGROUND

A. Technical Field

The present invention relates to a therapeutic device to promote the healing of a wound, comprising a cover around the wound to form an essentially airtight cover over the wound, whereby a liquid-permeable pad is preferably provided between the wound and cover, a drainage line connecting the cover with a suction pump, a drainage line connecting the cover with a suction pump so that the wound can be suctioned to draw fluids from it, and a connector to connect the cover with the drainage line, whereby the connector comprises an opening to connect the proximal end of the drainage line with the wound.

B. Background of the Invention

Such wound healing devices are used to treat secondary healing chronic or acute wounds, in particular in humans. Using the negative pressure present in the skin area around the wound is supposed to remove fluid, that is, in particular wound exudation, from the wound to thereby shorten the healing period. In many cases, the use of such a wound healing device makes wound healing possible in the first place.

A class-appropriate wound healing device is described in EP 1 088 569 B1. For the wound healing device disclosed in this patent it is in particular provided that the connector comprises a disc-like shell whose lower surface is in contact with a porous pad. This connector is obviously meant to insure even pressure distribution. The connector of the aforementioned wound healing device is provided for use in conjunction with a porous pad which is pressed onto the wound. The porous pad is extendible. An even distribution of the pressure on the wound is therefore problematic because the cross-section area of the drainage tube is generally distinctly smaller than the size of the wound.

It is therefore necessary to distribute the negative pressure applied by the drainage tube with or without use of a porous pad to the largest possible area so that pressure is applied as evenly as possible to the full dimension of the wound. To do so, the cited patent proposes that the connector be designed as a disk-like shell, whereby the lower surface of this shell is in contact with the porous pad.

In may case of practical application, however, this known shape of the connector is still not sufficient to guarantee a solid connection between the cover and the drainage tube. Moreover, the known disk-like shell is not always suitable for the respective shape of the wound as the provided curvature of the disk is sometimes not appropriate. The shape of a disk-like shell is however extendible to only a limited degree. For example, bending a convexly-bent disk-like shell to the wound can lead to a type of bulging of the surface for reasons of topology. Also, the disk-like shape of the shell of the connector is relatively rigid and as such cannot be adapted to the specific wound shapes or even to body shapes. These disadvantages result in limitations in the applicability of the known wound healing device.

### SUMMARY OF THE INVENTION

It is therefore the task of this invention to indicate a class-appropriate wound healing device to promote the healing of a wound, in which a more solid connection between the cover and the drainage line is guaranteed which however at the same time has a sufficient elasticity to conform to the respective body shape and/or wound shape. This task is inventively solved by the connector having fastening sections which extend from an area surrounding the opening to the area near the outer edge and are separated from each other by notches.

The fact that the fastening sections extend up to the proximity of the outer edge of the cover guarantees that the pressure is distributed from the relatively small outlet cross-section of the drainage tube to the broadest possible area of the wound. However, to avoid at such a large extension of the connector that it ceases to conform evenly to certain unfavorable body shapes and/or wound shapes, but rather maintains a certain distance from the wound and or the porous pad, notches are provided between individual fastening sections. These sections between individual fastening sections advantageously insure a certain elasticity of the connector even if, for example, the connector is produced from an essentially rigid material.

The advantage then consists of the combination of an increased application area of the connector compared to known connectors, on the one hand, with a simultaneous guarantee of a certain elasticity of the widened connector by notches, on the other.

The invention is further improved if the notches run in an essentially radial direction with respect to the axis of the opening. In particular, in the typical situation that a porous pad, such as an absorbent sponge, is placed on the wound, more or less convex surfaces are created on which the drainage tube has to be placed.

The bending of this surface can however vary depending on the part of the body on which the wound is located and the extent of the wound. If then according to the invention, the connector is to be widened compared to known connectors so that inventively it extends to the edge of the cover, this variability of the bending of the surface to be covered will increasingly become a problem, as explained below.

The inventive solution of this problem by distributing the connector into fastening sections is therefore especially effective if according to this design the notches run radially to the axis of the opening. For topological reasons, the deviation of a curved surface from a smooth surface becomes increasingly greater with increasing distance from a contact point of these two surfaces.

If therefore the notches also run radially to the axis of the opening, this deviation which is expressed in a separation/distance of the curved connector from the differently curved porous pad can therefore be most effectively avoided. By using radial notches it is possible for the fastening sections of the inventive connector to achieve a certain elasticity which would not exist without the notches, as unlike known connectors the inventive connector extends to the outer edge of the cover.

The effect of the inventive connector is further improved if the notches are designed as interruptions of an outer edge. The outer edge of the connector is found in the largest radial distance to the axis of the connector opening. As explained, the result of this is that a possible deviation of the connector's curve is at its maximum at that point from the curve of the porous pad. For this reason it is advantageous to interrupt these border areas in the form of notches.

If in a further embodiment of the invention, the notches are designed in a tapered fashion in the direction of the opening, the described radial enlargement of the curvature difference between the connector and the porous pad is considered in a particularly effective way. It is known that according to the state of the art the pressing together of the outer edge of a flexible disk-like shell topologically results in the bulging in the form of radially running folds. This problem is solved in a surprising simple way by the invention by the widening the notches with greater distance from the axis.

In a special embodiment of the invention, the connector is designed in an essentially star-shaped fashion. The symmetry of the star shape advantageously results in an especially even distribution of pressure across the wound.

An alternative embodiment of the inventive therapeutic device provides the notches in a spiral shape. In this case, a random number of spiral-like notches can be provided from the outer# edge of the connector in the direction of the axis of the opening. The already known problems are also solved in this case.

According to a variant of the present invention, the fastening sections form a cavity with respect to the wound. This results not only in a distribution of the pressure on the wound introduced by the drainage tube but also additionally an increase in the suction area. The suction of wound exudation can therefore occur additionally through the cavities under the fastening sections. An undesirable excessive remaining # of wound exudation in the wound or in a porous pad place thereon is thereby advantageously avoided.

The suction of wound exudation over a larger surface area is further optimized in a further development of the invention if the fastening sections are designed as tubes cut in an axial direction, whereby preferably a diameter of approximately ⅔ is cut away. The transport of wound exudation by tubes inventively cut open facing downwards works especially well since due to their edge-free interior contours the tubes are particularly well-suited for transporting fluids.

In order for the cavity between the open tubes and the wound and/or the porous wound dressing to not be excessively high and to still provide a sufficiently large cross section for suction, it has been shown to be especially favorable if approximately ⅔ of the diameter of the tubes is cut away. It goes without saying that the expression “cut away” does not necessarily refer to a production process for the inventive connection, but refers instead to the obtained result of a “third tube.” The third tube can for example also be produced by a casting process without requiring the removal of material.

An especially symmetrical pressure distribution is created in a special embodiment of the inventive therapeutic device if four fastening sections are provided.

Especially cost effective and also highly stable is an embodiment of the invention in which the connector is produced from a continuous injection-molded part. In this case, the fastening sections, the opening and an optionally provided connector piece for the drainage tube can all be produced as a continuous injection-molded part. This eliminates the need to assemble the individual components of the connector, which advantageously results in reducing costs.

Also, the embodiment as a continuous injection-molded part has the advantage of virtually eliminating leaks in the connector.

If a special embodiment of the invention provides an end of the opening turned away from the wound bent connecting sleeve, the forces between the tube and the connector created by the patient's movement artifacts are effectively absorbed.

In particular, the bending of the connecting sleeve makes it possible to run the drainage tube parallel to the patient's body so that the leverage at the connection site can be avoided which would be created if the drainage tube were connected vertically to the connector. Overall, the connection is advantageously more secure and robust.

A distribution of the pressure over the wound is achieved by the invention in a particularly effective manner if the connector is produced from an essentially rigid material. For example, the connector can be made of PVC or other plastics. Compared to the wound dressing and the cover and other components, these materials are rigid and can therefore be used as pressure distributors. On the other hand, these materials do exhibit a certain elasticity. This elasticity is particularly present at the ends of the fastening sections so that to a limited degree a “nestling” of the fastening sections of the inventive connector to the wound or the porous pad is achieved.

It is provided in a further development of the invention that a pressure sensor is supplied on at least one fastening section. As the connector is inventively provided in the negative pressure area of the wound, the fastening section is very well suited as a pressure measuring point. For example, a strain gage can be provided to measure pressure. The pressure measured in this way corresponds precisely to the local pressure on the wound which is crucial for vacuum therapy.

This measurement is particularly suited as influencing variable for a pressure adjustment of the vacuum therapy device. In this case it is especially advantageous if as a further development of the invention the pressure sensor is sealed in the fastening section.

This advantageously insures that the pressure sensor cannot detach from the connector and penetrate the wound. Also, the sealing of the pressure sensor rules out all contact of same with the wound itself or with the wound exudation. For a direct contact between the wound or the porous wound dressing it is advantageous in the embodiment of the invention if the connection is provided under the cover.

Alternatively, it is also conceivable to arrange the connector above the cover though this means that there is not direct contact between the connector and the wound and or porous pad, while on the other the fastening sections can no longer be used as pressure measuring points. Also, the suctioning through the fastening sections is no longer possible if the connector is provided under the cover.

According to a variant of the inventive therapeutic device, the pad comprises a natural sponge on which the connector is supported. Compared to the previously known use of synthetic materials as for example open-cell polymer plastics as a porous pad for vacuum therapy devices, natural sponges provide a number of advantages.

First of all, there is an increased demand by patients for natural or near-natural products. In addition, natural sponges have hemostatic properties in the form of compresses. Further, there are indications of biologically active ingredients, which have antibacterial, anti-inflammatory, cytotoxic or microstatic properties.

These many advantageous properties of natural sponges are also advantageous for vacuum healing therapy. Also, natural sponges are sufficiently available on the market in the desired quality for the use in a mass-produced commercial therapy device. The combination of a natural sponge with the inventive connectors for the even distribution of the fed pressure results in optimal care for the wound.

### BRIEF DESCRIPTION OF THE DRAWINGS

The invention is described in a preferred embodiment using a drawing, whereby other advantageous details can be found in the drawing figures.

Parts having the same function are labeled with the same reference numbers.

The figures of the drawings show:

FIG. 1 schematic representation of the inventive wound healing device and its operating mode

FIG. 2 perspective representation of one of the inventive connectors applied to the wound between the drainage tube and the wound cover

FIG. 3 individual representation of the inventive connector from a top view

FIG. 4a side view along the line A-A of the connector from FIG. 3

FIG. 4b perspective representation of a connector from FIG. 3 view from below

FIG. 5 principal representations of the shape of two variations of the inventive connector

### DETAILED DESCRIPTION OF THE PREFERRED EMBODIMENTS

The present invention also relates to a procedure for operating a class-appropriate therapy device.

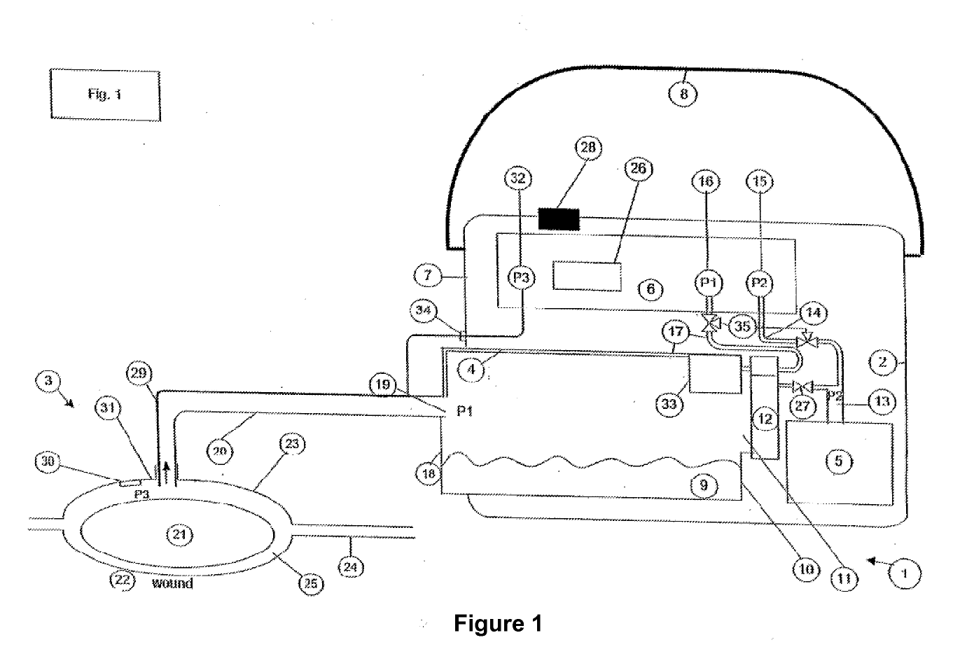

FIG. 1 schematically shows an embodiment of an inventive wound healing device 1. The wound healing device 1 comprises a base unit 2 and a wound dressing unit 3. The base unit 2 contains a replaceable collecting container 4, a suction pump 5 and a control device 6. The base unit 2 is enclosed by a housing 7. A carrying strap 8 is attached to the housing 7. The collecting container 4 contains a mixture 9 consisting of wound exudation and gelling agent.

The collecting container 4 comprises a suction opening 11 on one of the inner walls 10 facing the housing 7. The suction opening 11 is connected to the suction pump 5 by the filter 12 and a suction tube 13. A pump-side probe tube 14 branches off from the suction tube 13. The pump-side probe tube 14 carries the prevailing pressure p2 contained in the suction tube 13 to a suction-pump-side pressure sensor 15 which is located in the control device 6.

The control device 6 also contains the distal pressure sensor 16, which interconnects the prevailing pressure p1 prevailing on the distal end of the suction tube 17 via the probe tube 17 and the distal probe tube 36. A 3/2-way valve 35 is installed in both the probe tube 17 and in the probe tube 14. The distal probe tube 36 runs along the replaceable collecting container 4 inside the housing 7 to a suction opening 19 located in the outer wall 18 of the collecting container 4 in the housing 7 of the base unit 2. Also, the distal probe tube 36 can optionally be run through a black box 33.

The wound dressing unit 3 comprises the wound exudation suction tube 20, a sponge 21 and a film foil 23 through which the wound exudation suction tube 20 and the sponge 21 are fastened to skin 24 surrounding the wound 22 in such a way that an airtight connection is created.

The sponge 21 is able to absorb and store fluids. Using the stretched film foil 23, the sponge 21 is introduced with a certain contact pressure to the wound 22.

A connector 31 is provided between the sponge 21 and film foil 23. The drainage tube 20 is connected to the connector 31. The connector touches the side of the sponge 21 facing the wound 22.

Also, a strain gage 30 is fastened in the wound dressing 3 between the film foil 23 and the sponge 21 near the tube connector 31. The strain gage 30 is used to measure the wound pressure on the wound 22. The strain gage 30 is connected to an electrical cable 29.

The electrical cable 29 is connected to the outer edge of the suction tube 20 and in this way connected to the evaluation device 32 for the wound pressure P3 by means of a plug-type connector 34 in the housing 7 of the base unit 2. The evaluation device 32 is part of the control device 6.

The collecting container 4, the wound exudation suction tube 20, the sponge 21 and the film foil 23 are respectively sterile and replaceable for disposable use.

The wound exudation is located in the wound 22. To use the inventive wound healing device 1, the sponge 21 is introduced into the wound 22. Using the sponge 21, one end of the wound exudation suction tube 20 is connected via the connector 31, and then both the wound exudation suction tube 20, the connector 31 and the sponge 21 are affixed to the skin area 24 around the wound 22 with the help of the film foil 23 in such a way that the system is made airtight.

The other end of the wound exudation suction tube 20 is also connected to the suction opening 19 contained in the outer wall 18 of the collecting container 4 in an airtight fashion. Due to the property of the sponge 21 to absorb and store fluids, the wound exudation 25 is removed from the wound 22 into the sponge 21. The wound exudation quantity that exceeds the storage capacity of the sponge 21 is suctioned into the collecting container 4 through the wound exudation suction tube 20. The negative pressure needed for the suction is produced by the suction pump 5. For this purpose the suction tube 13 of the suction pump 5 interconnects the collecting container 4.

The filter 12 arranged between the collecting container 4 prevents the wound exudation 25 from entering the suction tube 13, the suction pump 5, the suction-pump-side probe tube 14 or even the suction-pump-side pressure sensor 15. The excess wound exudation 25 collected in the collecting container 4 in the manner described above is thickened by a gelling agent. This effectively prevents the moistening of the filter 12 even when the base unit 2 is moved. As a result, for example, the filter 12 is prevented from being moistened and germs are prevented from getting into the control device 6. Also, it is possible to dispose of the potentially contaminating wound fluid in the solid aggregate state in a more hygienic fashion.

The negative pressure produced by the suction pump 5 is continuously received on the one hand by the suction-pump-side pressure sensor 15 and by the wound-side strain gage 30 on the other and also via the distal pressure sensor 16, which measures the distal pressure p1. Then, the pressure p3 taken from the wound-side strain gage 30, the pressure p2 taken from the suction-pump-side pressure sensor 15 and the pressure p1 taken from the distal end of the suction tube 20 are used in the microprocessor of the control device 6 as input data for control.

In order to maintain a moist wound environment, the pressure level in the wound dressing unit 3 is set according to the individual needs of the patient, the properties of the wound 22 and according to the criteria of compatibility (pain by the patient).

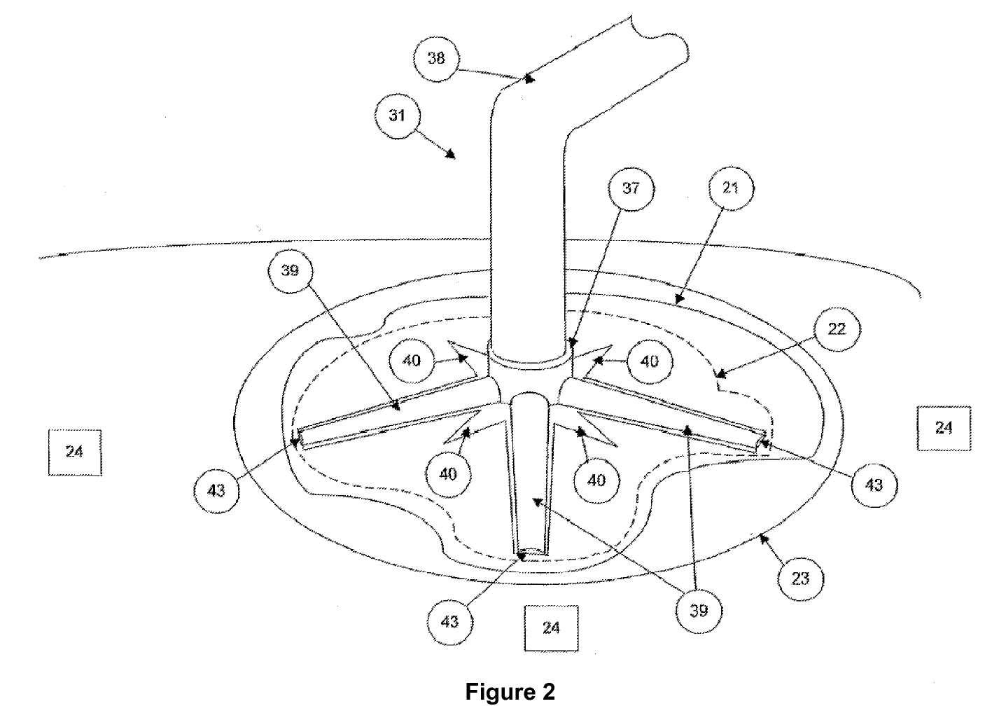

FIG. 2 shows the connector 31 in detail. The connector 31 is placed on the sponge 21. In turn, the sponge 21 completely covers the wound 22. The edge of the wound 22 is shown with a dotted line to indicate that the wound 22 is completely covered by the sponge 21 and therefore cannot be seen in this view.

The transparent film foil 23 is placed over the connector 31 and fastened to the skin 24 around the wound 22 in such a way that an essentially airtight seal is created. The connector 31 is designed as a continuous injection-molded part. For example, PVC (polyvinylchloride) is particularly suited as a material. However every other plastic or other material is suited that is used in medical therapy equipment and which is essentially rigid, with a certain elasticity.

The connector 31 is provided with an opening 37 on its upper end. A connecting sleeve 38 is placed on the opening 37. The connecting sleeve 38 is bent. An unspecified connection for the drainage tube 30 is located at the end of the connecting sleeve 38. The opening 37 of the connector 31 flows into four suction arms 39.

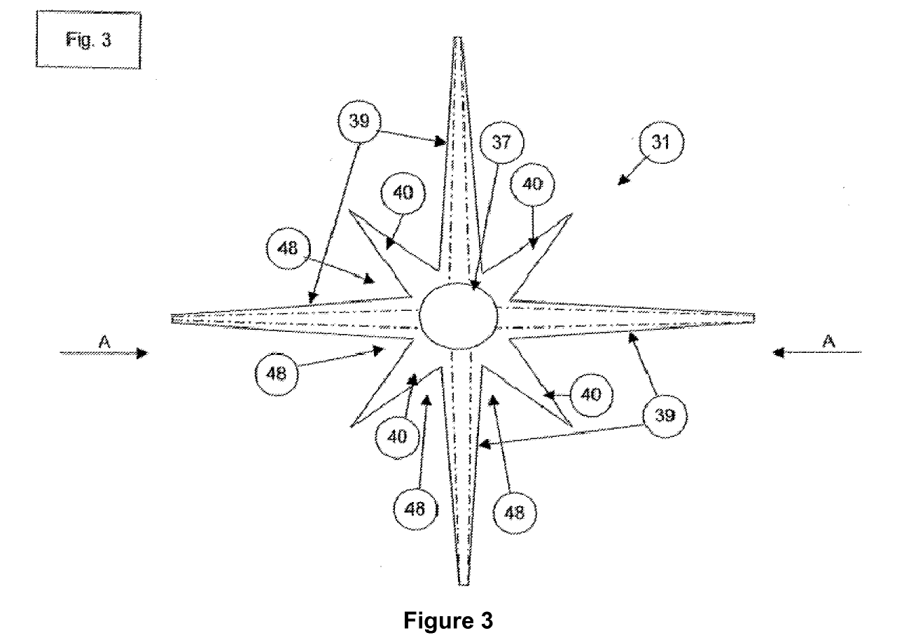

In FIG. 2 one of the suction arms 38 is hidden and can therefore not be seen. In addition, four fastening arms 40 are connected on the wound side to the opening 37 in the connector 31. The suction arms 39 are approximately three times as long as the suction arms 39. The double-star-like design of the connector 31 can also be seen in the top view in FIG. 3.

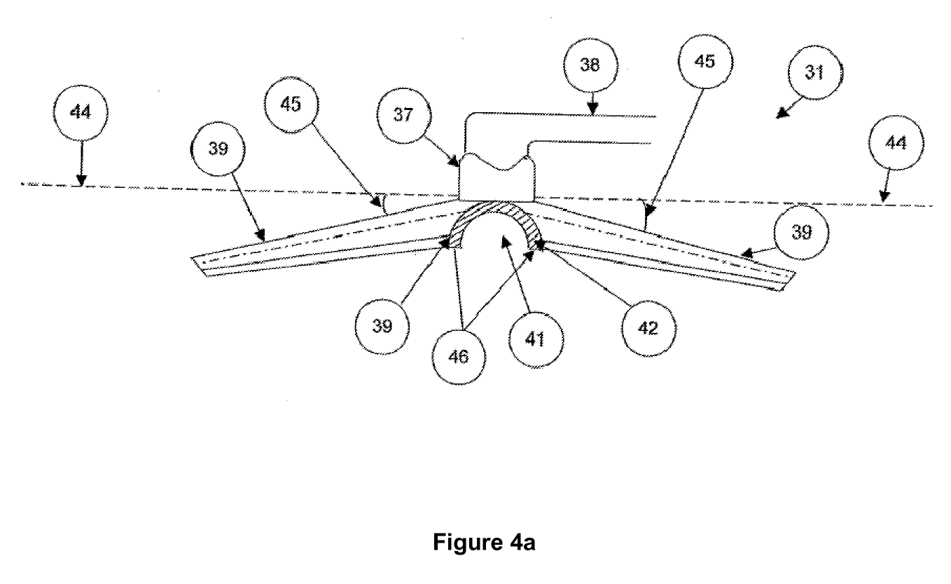

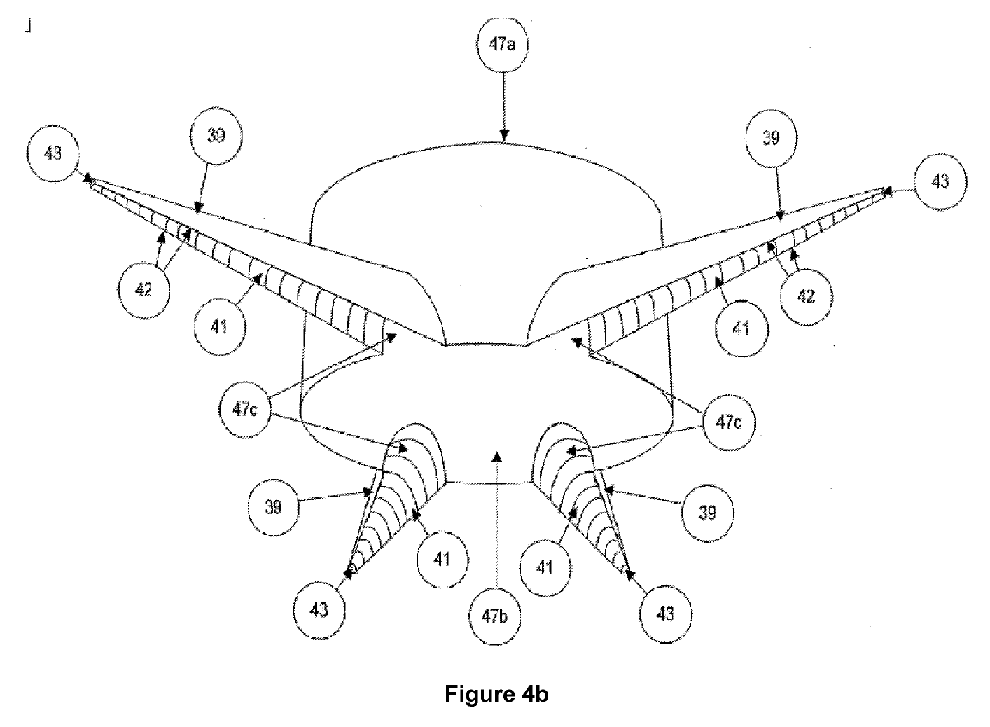

The suctions arms 39 have, as seen in FIG. 4, in cross section the shape of a tube cut open lengthwise. Each suction arm 39 therefore defines with its wall 42 a channel, which is turned toward the wound 22.

In the embodiment, both of the fastening arms 40 run toward the pointed end. The suction arms 39 extend to close to the edge of the film foil 23. The front area 43 of the suction arms 39 are closed. In this way the channel 41 is bordered upward by the wall 42 and at the end by the front area 43. However, the channel is open toward the bottom, i.e. toward the wound 22.

In the interest of improved clarity, the connecting sleeve 38 for the drainage tube 20 is not represented in FIG. 3. Unlike the suction arms 39, the fastening arms 40 are not convex and as such do not form a channel. Instead, the lower sides of the fastening arms 40 form a flat support surface which can be placed on the sponge 21.

As can be seen in FIG. 4, the suction arms 39 are bent around the wound at an angle 45. The fastening arms 40 are bent at the same angle 45 to the wound. The lower surfaces of the fastening arms 40 and the end surfaces 46 of the suction arms 39 therefore lie on an intentionally common curved surface of a cone.

When operating the wound dressing unit 3, the negative pressure applied to the drainage tube 20 by the suction pump 5 is applied to the wound with the assistance of the inventive connector 31. In this way the suction effect on the proximal end of the drainage tube 20 is distributed to the opening 37 of the connector 31 to the channels 41 of the four suction arms 39. As a result, the connector 31 is evenly pressed by the surrounding atmospheric pressure on the sponge 21. A shifting of the connector 31 on the sponge 21 is primarily prevented by the fastening arms 40 because the fastening arms 40 have a large contact surface on the sponge 21.

The connector 31 acts on the wound as a pressure distributor. The fastening arms 40 and in particular the suction arms 39 are somewhat elastic and can therefore adjust to a curvature of the sponge 21. The pressure is transmitted to near the edges of the film foil 23. The fastening arms 40 and in particular the suction arms 39 are somewhat elastic, and therefore can adjust to the bulge of the sponge 21. This insures a constant contact between the connector 31 and the sponge 21.

In addition to the pressure distributing effect, the efficiency of the suction of the wound exudation 25 which is stored in the sponge 21, is significantly improved. The improvement is achieved by a larger area, that is the opening area of the channels 41, being suctioned. The suction situation can be seen particularly well in FIG. 4b. It shows that the connector is provided with six outputs 47, specifically the upper outlet 47a, to which the drainage tube 20 is connected, which is opposite the outlet 47a, the lower outlet 47b which faces the wound, and the four outputs 47c which flow into the channels 41 of the respective suction arms 39.

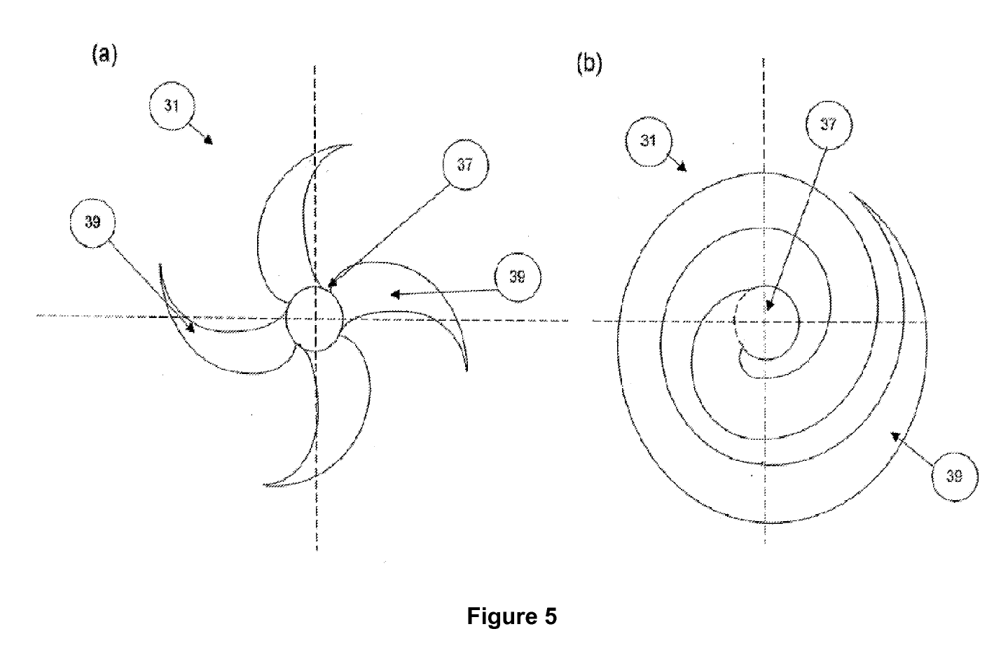

FIG. 5 shows alternative embodiments of the connector 31. The representations show the top view from FIG. 3 of the embodiment described above.

FIG. 5 shows an embodiment of the connector 31 according to the invention in which the suction arms 39 have a spiral shape. Part b of FIG. 5 shows another alternative embodiment of the connector according to the present invention in which only one spiral-shaped suction arm 39 is provided.

This suction arm winds a number of times around the opening 37. Although the task of the invention is also solved by a connector having the general shape according to FIG. 5b, this variant is not preferred for practical reasons. Instead, the preferred embodiment is the star-shaped construction depicted in FIGS. 2 through 4a/b.

### LIST OF DRAWING REFERENCES

1 Wound healing device
2 Base unit
3 Wound dressing unit
4 Collecting container
5 Suction pump
6 Control device
7 Housing
8 Carrying strap
9 Mixture
10 Inside wall
11 Suction opening
12 Filter
13 Suction tube
14 Suction-pump-side probe tube
15 Suction-pump-side pressure sensor
16 Wound-side pressure sensor
17 Wound-side probe tube
18 Outer wall
19 Suction opening
20 Wound exudation suction tube
20a Interior tube wall
20b Exterior tube wall
20c Cavity
21 Sponge
22 Wound
23 Film foil
24 Skin
25 Wound exudation
26 Display unit
27 Control valve
28 Computer interface
29 Electrical cable
30 Strain gage
31 Tube connector
32 P3 evaluation device
33 Black box
34 Plug-type connector
35 3/2-way valve
36 Distal probe tube
37 Opening
38 Connecting sleeve
39 Suction arm
40 Fastening arm
41 Channel
42 Wall
43 Front area
44 Horizontal
45 Angle
46 End surface
47a Outlet
47b Outlet
47c Outlet
47d Outlet
48 Notches

1. A therapeutic device to promote the healing of a wound, comprising:
a cover that is positioned around the wound and a liquid-permeable pad provided between the wound and the cover,
a drainage line coupling the cover with a suction pump that applies a suction to the wound to draw fluids from therefrom, and
a connector that couples the cover with the drainage line, the connector comprising an opening to couple a proximal end of the drainage line to the wound, wherein the connector comprises fastening sections which extend from at least one first area around the opening to an second area close to an outer edge of the cover and are separated from each other by a plurality of notches.

2. The therapeutic device according to claim 1 wherein the plurality of notches run essentially in a radial direction with respect to an axis of the opening.

3. The therapeutic device according to claim 1 wherein the plurality of notches are interruptions of an outer edge of the connector.

4. The therapeutic device according to claim 1 wherein the plurality of notches are tapered in the direction of the opening.

5. The therapeutic device according to claim 1, wherein the connector is essentially star-shaped.

6. The therapeutic device according to claim 1, wherein the plurality of notches have a spiral shape.

7. The therapeutic device according to claim 1, wherein the fastening sections form a cavity in a direction of the wound.

8. The therapeutic device according to claim 7, wherein the fastening sections comprise open tubes in an axial direction, and have diameters of approximately two-thirds cut away.

9. The therapeutic according to claim 1, wherein four fastening sections are provided.

10. The therapeutic according to claim 1, wherein the connector is produced from a continuous injection-molded part.

11. The therapeutic according to claim 1, wherein an end of the opening facing away from the wound has a bent connecting sleeve (38).

12. The therapeutic according to claim 1, wherein the connector is constructed of essentially rigid material.

13. The therapeutic according to claim 1, wherein a pressure sensor is installed on at least one of the fastening sections.

14. The therapeutic device according to claim 13, wherein the pressure sensor is sealed in the fastening section.

15. The therapeutic according to claim 1, wherein the connector is provided under the cover.

16. The therapeutic according to claim 1, wherein the pad comprises a natural sponge which supports the connector.
